(19) United States
(12) Patent Application Publication
(10) Pub. No.: US 2006/0105344 A1
(43) Pub. Date: May 18, 2006

(54) Modulation of deubiquitinase family members

(76) Inventors: Rene Bernards, Amsterdam (NL); Annette M Dirac, Amsterdam (NL); Senastian M Nijman, Amsterdam (NL)

Correspondence Address: NIXON & VANDERHYE, PC, 901 NORTH GLEBE ROAD, 11TH FLOOR, ARLINGTON, VA, 22203, US

(21) Appl. No.: 10/542,024

(22) PCT Filed: Jan. 16, 2004
(86) PCT No.: PCT/EP04/00408

§ 371 Date: Sep. 12, 2005

(30) Foreign Application Priority Data
Jan. 17, 2003 (GB) 0301124.4
Jun. 12, 2003 (GB) 0313625.6

(51) Int. Cl. C12Q 1/68; G01N 33/567; A61K 31/60; A61K 31/557

(52) U.S. Cl. 435/6; 435/7.2; 514/165; 514/573

(57) ABSTRACT

Modulation of Deubiquitinase Family Members The present invention relates to the therapeutic treatment of conditions by modulating members of the deubiquitinase family, and also to assays for identifying substances which may be useful in such treatments, as well as to the treatment of cylindromatosis and more generally to the modulation of other conditions associated with activation of the transcription factor NF-κB, such as inflammation.

### FIELD OF THE INVENTION

The present invention relates to the therapeutic treatment of conditions by modulating members of the deubiquitinase family, and also to assays for identifying substances which may be useful in such treatments.

In one aspect, the invention relates generally to assay methods for identifying modulators of HIF-α, where the assay involves identifying substances which bind to and/or modulate an activity of VDU1. It also relates to modulators of VDU1 for use in methods of medical treatment, and in particular, in the treatment of conditions which can be improved by modulating the activity of HIF.

In another aspect, the invention relates to the treatment of cylindromatosis and more generally to the modulation of other conditions associated with activation of the transcription factor NF-κB, such as inflammation.

### BACKGROUND TO THE INVENTION

HIF

The transcription factor HIF (hypoxia inducible factor) system is a key regulator of responses to hypoxia, occupying a central position in oxygen homeostasis in a wide range of organisms. A large number of transcriptional targets have been identified, with critical roles in angiogenesis, erythropoiesis, energy metabolism, inflammation, vasomotor function, and apoptotic/proliferative responses. The system is essential for normal development, and plays a key role in pathophysiological responses to ischaemia/hypoxia. HIF is also important in cancer, in which it is commonly up-regulated, and has major effects on tumour growth and angiogenesis.

The HIF DNA binding complex consists of a heterodimer of α and β subunits. Regulation by oxygen occurs through the α-subunits, which are rapidly destroyed by the proteasome in oxygenated cells. This involves targeting of HIF-α subunits by the von Hippel-Lindau tumour suppressor (pVHL), with pVHL acting as the recognition component of a ubiquitin ligase that promotes ubiquitin dependent proteolysis through interaction with a specific sequence in HIF-α-subunits. In hypoxia, this process is suppressed, so stabilizing HIF-α and permitting transcriptional activation.

CYLD

Cylindromas are rare benign adnexal tumours that arise primarily on the scalp in humans. They occur at any age but usually appear in early adulthood. There are 2 distinct clinical forms: a solitary form, which is sporadic, and a multiple form, which is dominantly inherited, referred to as familial cylindromatosis. The lesions are pink-red, nodular, firm, and usually painless, and vary in size from several millimetres to more than 6 cm in diameter. The tumours grow slowly in size and number throughout life; in severe cases, they may cover the entire scalp and are known as “turban tumours.” At present, treatment is by surgery to remove the tumours followed by reconstruction of the affected area.

The condition has been linked to the chromosomal region 16q12-13 and more recently Bignell, G. R. et al., (Nat Genet 25, 160-5 (2000)) identified the gene, CYLD, in this region which is mutated in individuals with cylindromatosis. The gene is regarded as a tumour suppressor gene though its mode of action has not been elucidated.

Nuclear factor κB (NF-κB) is a sequence-specific transcription factor that is known to be involved in the inflammatory and innate immune responses. NF-κB is activated by release from an inhibitory factor, referred to as IkB. NF-κB is a heterodimer consisting of a 50 kDa (p50) and a 65 kDa (p65) DNA-binding subunit. NF-κB contributes to the so-called “immediate-early” activation of defence genes if cells are exposed to primary or secondary pathogenic stimuli.

It has also been found that NF-κB and the signalling pathways that are involved in its activation are also important for tumour development, in that NF-κB also regulates cell proliferation and apoptosis. NF-κB has been shown to be constitutively activated in several types of cancer cell.

### DISCLOSURE OF THE INVENTION

The present inventors have identified a regulatory pathway present in cells in which HIF-α is stabilised by the action of the VHL-interacting deubiquitinase enzyme 1 (VDU1).

Previously, no target for deubiquitination by VDU1 has been identified and so no physiological role has been demonstrated.

The activity of VDU1 represents a novel target for the control of HIF-α. Loss of VDU1 leads to a decrease in HIF-α, and a reduction in the responses mediated by HIF. Accordingly, reduction of VDU1 may be useful in the treatment of diseases associated with inappropriate HIF activity, or in other conditions in which reduction of HIF activity can have some therapeutic benefit.

Conversely, it is believed that increasing VDU1 activity will stabilise HIF-α and lead to an increase of HIF-mediated responses, which may be beneficial for example in promoting new vascular growth.

The finding that HIF-α stability can be regulated by VDU1 provides for a novel assay method for the development of new agents for human or animal therapy.

In one aspect, assays of the invention are generally directed at determining whether a test substance is capable of modulating the stability and/or state of ubiquitination of HIF-α, via modulation of VDU1.

This may be done by:

bringing into contact a putative modulator and a VDU1 polypeptide;

determining whether the putative modulator binds and/or modulates an activity of VDU1;

determining the effect of the putative modulator on HIF-α stability and/or on the ubiquitination state of HIF-α in a test system comprising HIF-α and VDU1.

Accordingly, in one aspect, the invention provides an assay method which includes:

bringing into contact a VDU1 polypeptide with a putative modulator;

determining binding between the VDU1 polypeptide and the putative modulator;

bringing the putative modulator into contact with a test system comprising VDU1 and HIF-α; and

determining the effect of the putative modulator on the stability and/or state of ubiquitination of HIF-α.

It is known that VDU1 and HIF-α both bind to the β-domain of VHL, and that VHL targets both these proteins for ubiquitination (Li et al, 2002). In the light of the present finding that VDU1 stabilises HIF-α, it is believed that the binding of VDU1 to VHL may bring VDU1 into physical proximity with HIF-α and so may facilitate the interaction between VDU1 and HIF-α.

Also, it is believed that an agent which blocks the ability of VDU1 to bind VHL may reduce the extent to which HIF-α is stabilised by VDU1, and/or may alter the state of ubiquitination of HIF-α, and hence reduce the activity of HIF in the cell.

Accordingly, in another aspect of the invention, there is provided an assay method which includes:

bringing into contact a VHL polypeptide, a VDU1 polypeptide and a putative modulator;

determining whether the putative modulator modulates the interaction of the VHL and VDU1 polypeptides;

bringing the putative modulator into contact with a test system comprising VDU1, VHL and HIF-α;

determining the effect of the putative modulator on the stability and/or state of ubiquitination of HIF-α.

In this aspect, the relevant activity of VDU1 is its ability to bind VHL.

In a further aspect of the invention, there is provided an assay method comprising:

bringing a putative modulator into contact with VDU1 and an ubiquitinated VDU1 substrate;

determining the ability of the putative modulator to modulate the stabilisation and/or state of ubiquitination of the substrate by VDU1;

bringing the putative modulator into contact with a test system comprising VDU1 and HIF-α;

determining the effect of the putative modulator on the stability and/or state of ubiquitination of HIF-α.

In this aspect, the relevant activity of VDU1 is the ability to deubiquitinate and stabilise a substrate.

Determining whether a test substance is capable of modulating the stability and/or state of ubiquitination of HIF-α via modulation of VDU1 can also be done, in further aspect of the invention, by:

bringing into contact a putative modulator with a test system comprising VDU1 and ubiquitinated HIF-α;

determining the ability of the putative modulator to modulate the stabilisation and/or state of ubiquitination of HIF-α by VDU1.

In the assays above, the test system can optionally comprise VHL, especially when the assay is an assay for an inhibitor.

Specific modulators of VDU1 have not previously been shown to be useful for methods of therapy. Accordingly, the invention also provides a modulator of VDU1 for use in a method of medical treatment, and, in another aspect, a composition comprising a modulator of VDU1 and a pharmaceutically acceptable excipient.

In a further aspect, the invention provides the use of a modulator of VDU1 for the manufacture of a medicament for the treatment of a condition in which modulation of HIF is of therapeutic value.

In a still further aspect, the invention provides a method of treating a disease in which modulation of HIF is of therapeutic value, the method comprising administering to an individual an effective amount of an agent which modulates the activity of VDU1.

The present inventors have also identified a regulatory pathway present in cells which directly links the action of CYLD to the suppression of NF-κB. Loss of CYLD thus leads to an increase in NF-κB activity, which in turn causes an increase in anti-apoptopic gene function. This may result in the disruption of the balance of pro- and anti-apoptopic gene regulation in cells of the skin, leading to the growth of the benign tumours associated with cylindromatosis.

Thus in another aspect, the invention provides a method of treating an individual with cylindromatosis by administering to the individual an effective amount of an NF-κB inhibitor.

In further aspect, the invention provides the use of an NF-κB inhibitor for the manufacture of a medicament for the treatment of cylindromatosis. Alternatively, the invention provides an NF-κB inhibitor for use in a method of treatment of cylindromatosis.

In another aspect, the finding that the action of CYLD is to suppress NF-κB activity provides a new target for the treatment of diseases associated with activation of NF-κB. Thus the invention provides a method of treating such a disease in an individual by administering to the individual an effective amount of an agent which increases expression of CYLD. In another aspect, the invention provides the use of an agent which increases expression of CYLD for the manufacture of a medicament for the treatment of a disease associated with activation of NF-κB. Alternatively, the invention provides an agent which increases expression of CYLD for use in a method of treatment of a disease associated with activation of NF-κB.

In a further aspect, the finding that NF-κB activity can be regulated by CYLD provides a novel assay method for the development of new agents for human or animal therapy. Thus the invention provides an assay method which includes the steps of:

providing a cell culture in which CYLD activity is suppressed or missing;

bringing the culture into contact with an agent to be assayed; and

determining the effect of the agent on the activity of NF-κB.

These and other aspects of the invention are described further herein below.

### DESCRIPTION OF THE DRAWINGS

FIG. 1—Inverse values of the 3×RE-luciferase HIF-1α responsive reporter and individual members of the DUB knockdown library under conditions that mimic hypoxia in HEP-G2 cells (12 hrs 1 mM. Desferrioxamine (DFO) exposure starting 48 hrs after transfection). SV40 Renilla luciferase served as an internal control.

FIG. 2—Activity of the 3×RE luciferase reporter in the presence and absence of pSUPER-VDU-1 under normoxic conditions and conditions that mimic hypoxia (12 hrs DFO). SV40 Renilla luciferase served as an internal control.

FIG. 3—U2-OS cells were transfected with pSUPER or pSUPER-VDU-1 and exposed to 1 mM DFO for 12 hrs. Whole cell extracts were immunoblotted with a Hif1-α specific antibody.

FIG. 4 CYLD is an antagonist of NF-κB signalling. U2-OS cells were transfected with a NF-κB luciferase reporter and pSUPER-CYLD or empty vector. Forty-eight hours after transfection cells were stimulated overnight with PMA (200 nM) or TNF-α (20 ng/ml) and luciferase activity was measured. SV40 Renilla luciferase served as an internal control.

### DETAILED DESCRIPTION OF THE INVENTION

HIF

The amino acid sequence of human VDU1 is given in Li et al (2002), and is also given in Genbank reference AF383172. At least two putative subtypes are known. Type I consists of 942 amino acids and type II consists of 911 amino acids with predicted molecular masses of 107 and 103 kDa, respectively. In the present application, VDU1 will be understood to be any suitable mammalian VDU1, preferably human VDU1, including alleles, homologs and orthologs of the known sequences. Although wild type sequences are preferred, the term VDU1 will also be understood to include variants, provided they retain the ability to deubiquitinate HIF-α. Preferably, the variants also retain the ability to bind VHL.

Generally it is preferred that variants have a degree of amino acid identity which is desirably at least 70%, preferably at least 80%, 90%, 95% or even 98% to a wild type mammalian VDU1, preferably to human VDU1.

VHL has also been cloned, and the sequence of human VHL is available as Genbank accession numbers AF010238 and L15409.

A number of HIF-α subunit proteins have been cloned. These include HIF-1α, the sequence of which is available as Genbank accession number U22431; HIF-2α, available as Genbank accession number U81984; and HIF-3α, available as Genbank accession numbers AC007193 and AC079154. These are all human HIF-α subunit proteins.

In the present application, VHL and HIF-α will be understood to be any suitable mammalian VHL or HIF-α, preferably human, including alleles, homologs and orthologs of the known sequences. HIF-α is preferably HIF-1α.

Although wild type proteins are preferred, references to the HIF-α and VHL in these assay methods also include reference to variants and fragments which retain a relevant function of the wild type protein. In the assay methods discussed herein, suitable VHL variants are those which retain the ability to bind HIF-α and VDU1. More preferably, they also retain the ability to target HIF-α for ubiquitination. Suitable HIF-α variants preferably retain the ability to be labelled with ubiquitin, and to be recognised for de-ubiquitination by VDU1. More preferably, they also retain the ability to bind VHL. In some embodiments, they retain the ability to bind to and/or activate a response element.

Generally it is preferred that variants have a degree of amino acid identity which is desirably at least 70%, preferably at least 80%, 90%, 95% or even 98% to a wild type mammalian HIF-A or VHL, preferably to human HIF-α or VHL.

Sequence identity can be assessed using the algorithm BLAST 2 SEQUENCES, using default parameters.

Assay Methods

Assay methods of the invention in this aspect provide modulators of VDU1 activity which are useful in treating conditions in which HIF activity is harmful or may be beneficial. These conditions are discussed in more detail below.

In some aspects of the invention, the assay methods involve a first stage of assessing whether the putative modulator binds to a VDU1 polypeptide, or affects the interaction between VDU1 and VHL polypeptides.

In respect of the first stage of these assay methods, it will be understood that the term “VHL polypeptide” or “VDU1 polypeptide” includes reference to VDU1 and VHL as defined above, but also includes fragments of these proteins. Generally fragments, where used, will be at least 40, preferably at least 50, 60, 70, 80 or 100 amino acids in size. Where the assay involves the assessment of binding between VDU1 and VHL, then fragments of any size may be used, provided that they retain the ability to bind to each other in the absence of a test compound.

Preferred VHL fragments for use in assessing the VDU1/VHL interaction include those which are based at least in part upon the beta domain located within the fragment 63-156 of the 213 amino acid human VHL protein, or the equivalent domain in other variants. In a preferred embodiment, such domains will have at least 70%, preferably 80%, 90%, 95% or even 98% degree of sequence identity to the 64-156 fragment of human VHL. Fragments of this region and its variants may be used. These fragments may be 15-80 amino acids in length, for example from 20 to 80, such as 30-60 amino acids in length. Desirably, the wild-type sequence of the beta domain is retained.

Fragments may include the region 63-83 of human VHL or their equivalents in the above described variants.

One fragment which may be used is that in which up to 53 of the N-terminal residues, e.g. from 1 to n wherein n is an integer of from 2 to 53, have been deleted, the rest of the protein being wild-type.

Fragments may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art.

Where the assay method includes bringing into contact a VHL polypeptide, a VDU1 polypeptide and a putative modulator, and determining whether the putative modulator modulates the interaction of the VDU1 and VHL polypeptides (i.e., in the second aspect of the invention described above), it will also be understood that the terms “VDU1 polypeptide” and “VHL polypeptide” are also intended to refer to variants of VHL or VDU1 other than those described above, provided they retain the ability to bind VDU1 or VHL, respectively.

The step of bringing into contact a VHL polypeptide, a VDU1 polypeptide and a putative modulator compound may be done under conditions where the VHL polypeptide and the VDU1 polypeptide, in the absence of modulator, are capable of forming a complex.

In an alternative embodiment, the step may be carried out in conditions where the association does not occur in the absence of the modulator. This may be desirable for looking for agents which enhance or potentiate the binding.

Determining the effect of the putative modulator on the binding of VHL and VDU1 may be done in the presence and absence of the modulator. A change, i.e. an increase or decrease in binding in the presence relative to the absence of the putative modulator will be indicative of the ability of the putative modulator to modulate the interaction.

Generally, the method of determining binding or modulation of complex formation is not part of the present invention and the skilled person may use any of the methods known in the art.

To identify the binding of small molecules to polypeptides, standard assay formats may be used. For example, the polypeptide may be immobilised on a support, and a known amount of small molecule or a detectably labelled small molecule may be added to the protein. The interaction may be measured, for example, as described below in relation to in vitro assays for protein-protein interaction.

One assay format which is widely used in the art to study the interaction of two proteins is a two-hybrid assay. This assay may be adapted for use in the present invention. A two-hybrid assay comprises the expression in a host cell of the two proteins, one being a fusion protein comprising a DNA binding domain (DBD), such as the yeast GAL4 binding domain, and the other being a fusion protein comprising an activation domain, such as that from GAL4 or VP16. In such a case the host cell (which again may be bacterial, yeast, insect or mammalian, particularly yeast or mammalian) will carry a reporter gene construct with a promoter comprising DNA binding elements compatible with the DBD. The reporter gene may be a reporter gene such as chloramphenicol acetyl transferase, luciferase, green fluorescent protein (GFP) and β-galactosidase, with luciferase being particularly preferred.

Two-hybrid assays may be in accordance with those disclosed by Fields and Song, 1989, Nature 340; 245-246. In such an assay the DNA binding domain (DBD) and the transcriptional activation domain (TAD) of the yeast GAL4 transcription factor are fused to the first and second molecules respectively whose interaction is to be investigated. A functional GAL4 transcription factor is restored only when two molecules of interest interact. Thus, interaction of the molecules may be measured by the use of a reporter gene operably linked to a GAL4 DNA binding site which is capable of activating transcription of said reporter gene.

Thus two hybrid assays may be performed in the presence of a potential modulator compound and the effect of the modulator will be reflected in the change in transcription level of the reporter gene construct compared to the transcription level in the absence of a modulator.

Host cells in which the two-hybrid assay may be conducted include mammalian, insect and yeast cells.

The interaction of VHL and VDU1 may also be examined directly, for example using microcalorimetry.

Another assay format measures directly the interaction between VDU1 and VHL by labelling one of these proteins with a detectable label and bringing it into contact with the other protein which has been optionally immobilised on a solid support, either prior to or after proteins have been brought into contact with each other. Suitable detectable labels include ³⁵S-methionine which may be incorporated into recombinantly produced proteins, and tags such as an HA tag, GST or histidine. The recombinantly produced protein may also be expressed as a fusion protein containing an epitope which can be labelled with an antibody. Alternatively, an antibody against VDU1/VHL can be obtained using conventional methodology.

The protein which is optionally immobilized on a solid support may be immobilized using an antibody against that protein bound to a solid support or via other technologies which are known per se.

Alternatively, the interaction of the proteins may be measured by immunoprecipitation of one followed by immunological detection of the other, e.g. by western blotting or electrophoretic mobility of detectably labelled proteins.

In a further alternative mode, one of VDU1 and VHL may be labelled with a fluorescent donor moiety and the other labelled with an acceptor which is capable of reducing the emission from the donor. This allows an assay according to the invention to be conducted by fluorescence resonance energy transfer (FRET). In this mode, the fluorescence signal of the donor will be altered when VDU1 and VHL interact. The presence of a candidate modulator compound which modulates the interaction will increase or decrease the amount of unaltered fluorescence signal of the donor.

FRET is a technique known per se in the art and thus the precise donor and acceptor molecules and the means by which they are linked to VDU1 and VHL may be accomplished by reference to the literature.

Suitable fluorescent donor moieties are those capable of transferring fluorogenic energy to another fluorogenic molecule or part of a compound and include, but are not limited to, coumarins and related dyes such as fluoresceins, and suitable acceptors include, but are not limited to, coumarins and related fluorophores, and the like.

Another technique which may be used is a scintillation proximity assay (reagents and instructions available from Amersham Pharmacia Biotech) in which a target compound (i.e. for this invention VHL, VDU1) is held on (or in the course of the assay attached to) a bead having a signalling compound which scintillates when activated by radioactivity emitted by a radiolabel attached to a target-binding molecule (i.e. for this invention another of VHL or VDU1).

In another aspect of the invention, the assay comprises the first step of bringing a putative modulator into contact with VDU1 and an ubiquitinated VDU1 substrate, and determining the ability of the putative modulator to modulate the stabilisation and/or state of ubiquitination of the substrate by VDU1.

An “ubiquitinated substrate” herein refers to a molecule conjugated to one or more ubiquitin moieties, which is a substrate for deubiquitination, e.g., by VDU1.

Ubiquitinated VDU1 substrates which may be used in the above methods include for example ubiquitinated GST or ubiquitinated beta-galactosidase.

This first step is preferably carried out in vitro. In this embodiment, the ubiquitinated substrate may be labelled, e.g, with [³⁵S] methionine.

The ubiquitinated substrate may in some embodiments be provided by the presence of an active ubiquitination system, but in other embodiments, ubiquitinated substrate may be provided, e.g., by isolation of the substrate from a cell or by in vitro ubiquitination.

Alternatively, the first step of this assay may be carried out in a cell expressing the substrate (optionally a labelled substrate), preferably a mammalian cell line, more preferably a human cell line.

Methods of determining the ability of a putative modulator to modulate the stabilisation of a substrate by VDU1 are discussed below, in relation to HIF-α. Unless otherwise apparent from the context, these methods also apply to other VDU1 substrates. Certain preferred formats for determining the ability of the modulator to modulate the stabilisation of the substrate will be apparent from this discussion.

In the aspects of the invention discussed above, the assay method also includes a step of contacting a test system comprising HIF-α and VDU1 with the modulator, and determining the effect on HIF-α stability and/or on the ubiquitination state of HIF-α. In some embodiments, VHL is also present in the test system.

The test system could be an in vitro test system. In one embodiment, the test system may comprise labelled HIF-α, e.g., labelled with [³⁵S]methionine. The test system may also comprise cell extract, which may be the source of the VDU1, HIF-α and/or VHL. The cell extract can be obtained from any cell, and is preferably obtained from one of the cell lines described below.

In order to assess the effect of the deubiquitinase, the HIF-α in the test system is preferably conjugated to one or more ubiquitin moieties, at least transiently. This may in some embodiments be achieved by the presence of VHL and other components of the ubiquitination system, together with free ubiquitin. In other embodiments, ubiquitinated HIF-α may be provided, e.g., by isolation of HIF-α from a cell, especially a normoxic cell, or by in vitro ubiquitination. In vitro ubiquitination of HIF-α may be achieved using a reconstituted complex of VHL, Rbx1, Cul2, Elongin B and Elongin C (see for example Kamura et al 2000, PNAS vol. 97, no. 19: 10430-10435).

In another embodiment, the test system could be a cell. Assays according to the invention may be performed in any cell line expressing HIF-α, preferably one in which the HIF-α ubiquitination system is active, preferably a mammalian cell line, more preferably a human cell line.

In some embodiments, the cell line may express a labelled version of HIF-α, e.g., labelled with a histidine tag, to allow isolation of the protein.

In some embodiments of the invention, the cell may be under hypoxic conditions. Under these conditions the HIF pathway will be at a high level of activation. This may be preferred for example when the modulator is an inhibitor of VDU1, and so decreases the stability of HIF-α.

In other embodiments of the invention, the cell may be under normoxic conditions. Under normoxic conditions, the HIF pathway will generally be at a low level of activation. This may preferred for example when the modulator is an activator of VDU1, and so will increase the stability of HIF-α.

The stability of HIF-α in an assay method of the invention may be determined by a variety of means. For example, where the effect of the test compound on HIF activity is assessed in vitro, the effect may be assessed by determining the level of ubiquitination of HIF-α, for example by determining the change in molecular weight or by isolating HIF-α (e.g., by immunoprecipitation) and then immunoblotting with antibodies against ubiquitin. Where the effect of the test compound is assessed in a cell, it is also possible to assess the amount of HIF-α in the cell, e.g., using Western blotting. Additionally, the activity of HIF-α can be examined using a reporter gene assay (e.g., firefly luciferase, secreted alkaline phosphatase or green fluorescent protein) whose promoter comprises a target site recognised by HIF, e.g., a promoter from the VEGF or erythropoietin genes.

It is preferred that determining the effect of the modulator on the stability of HIF-α will be carried out under conditions where VDU1 is capable of stabilising HIF-α in the absence of the modulator. This is particularly preferred where the assay is for inhibitors of VDU1 activity.

In an alternative, the assay may be carried out under conditions where VDU1 cannot stabilise HIF-α in the absence of the modulator, which may be desirable for example when the assay is an assay for an activator.

Determining the effect of the putative modulator on the stability of HIF-α may be done in the presence and absence of the modulator. A change, i.e. an increase or decrease in HIF-α stability in the presence relative to the absence of the putative modulator will be indicative of the ability of the putative modulator to modulate HIF-α stability.

Because the assays above comprise a preliminary step of assessing binding to VDU1 or modulation of an activity of VDU1, then it will be apparent that, e.g., changes in reporter gene expression are due to a change in the action of VDU1 on HIF-α and hence represent a change in stability. However, it may be preferred that stability is measured directly, e.g., by measuring a change in the amount of protein or more preferably by measuring a change in ubiquitination state of HIF-α, as below.

In an alternative aspect of the invention, the assay method includes:

bringing into contact a putative modulator with a test system comprising VDU1 and ubiquitinated HIF-α;

determining the ability of the putative modulator to modulate the stabilization and/or state of ubiquitination of HIF-α by VDU1.

The test system may be a test system as described above, although certain preferred embodiments will be apparent from the following discussion.

In this assay, it is necessary to determine the ability of the modulator to modulate the stabilisation of HIFα by VDU1, rather than, for example, the ability to directly affect HIF-α, or to affect the ubiquitination pathway. This can be done by eliminating the other possibilities, in various ways as will be apparent to the skilled person in the light of the present disclosure.

The following discussion applies also to determining the ability of a putative modulator to modulate the stabilisation of other ubiquitinated VDU1 substrates, as discussed above.

For example, in order to eliminate the possibility that the modulator is acting on the ubiquitination pathway rather than the deubiquitination pathway, the assay may be carried out under conditions where the ubiquitination pathway is not active. This may be achieved by carrying out the assay in the absence of a factor, e.g., a protein, which is required for substrate ubiquitination. The absence may be a total absence from the system, or may be absence in a functional form. For example, where the ubiquitinated substrate is HIF-α, the assay may be carried out in the absence of a component of the E3 ubiquitin ligase, such as elongin C, elongin B, cullin-2 or rbx-1. In some embodiments, the assay may be carried out in the absence of VHL.

Where no ubiquitination activity is present, the substrate will need to be provided in an ubiquitinated form. This may be done for example by in vitro ubiquitination, or by isolating the substrate from a cell. In the case of HIF-α, this is especially a cell under normoxic conditions. The most convenient format for such an assay will be in vitro. The test system may comprise cell extract in some embodiments, e.g., an extract from a cell deficient in a relevant ubiquitinase activity (such as a HIF-α ubiquitinase activity).

Methods of assessing HIF-α stability are described above, and these methods may also be used to determine the ability of the putative modulator to modulate the stabilisation of HIF-α (or, where applicable, other ubiquitinated VDU1 substrate) by VDU1.

In order to exclude the possibility that the modulator interacts directly with HIF-α (or, where applicable, other substrates), the assay preferably involves directly assessing the ubiquitination state of the substrate, e.g., by detecting a change in molecular weight or by immunoblotting with antibodies against ubiquitin.

Another method of confirming that the test substance is able to modulate the stabilisation of HIF-α (or other substrates) by VDU1 is to carry out control experiments, which the skilled person will be able to design in the light of the present disclosure using his general skill and knowledge. For example, in order to confirm that a test substance is modulating deubiquitination and not, for example, modulating the ubiquitination pathway, it would be possible for the skilled person to take a different test system, comprising the ubiquitination system and a substrate thereof (but without a functional deubiquitination pathway), and determine whether the putative modulator can modulate the ubiquitination state of the substrate in that system. Similarly, to confirm that the effect is not due to a direct modulation of HIF-α, it would be possible to take a test system comprising HIF-α and a reporter gene, and to determine whether the modulator has any effect in this test system. Other control experiments will be apparent to the skilled person.

In each of the assay methods of the invention described above, the amount of agent which may be used will normally be determined by trial and error. Typically, from about 1 nm to 100 μm concentrations of agent compounds may be used, for example from 0.1-10 μm. Agent compounds which may be used may be natural or synthetic compounds used in drug screening programs. Extracts of plants or microorganisms which contain several characterised or uncharacterised components may also be used.

Therapeutic Methods and Uses.

As indicated above, HIF is a transcription factor having a number of known transcriptional targets. There are therefore a number of diseases in which reduction or enhancement of HIF activity may be of value in treatment.

Diseases in which reduction of HIF activity may be of value may be those associated with HIF activity, more preferably with HIF1 activity. Preferably, they are diseases which are associated with inappropriate angiogenesis or with inflammation. Specific examples include cancer (see for example Cramer et al, 2003), eye diseases such as macular degeneration and diabetic retinopathy (Witmer et al, 2003), Alzheimer's (Vagnucci et al, 2003), atherosclerosis (Ross J S et al, 2001), psoriasis (Dredge et al, 2002), rheumatoid arthritis (Dredge et al, 2002) endometriosis (Healy et al, 1998), and the like.

Enhancement of HIF activity may be useful for example when new vascular growth and/or promotion of cellular survival or cellular function in hypoxia is of benefit, e.g., in peripheral or coronary artery diseases (Kusumanto et al, 2003) or in myocardial ischaemia and the like. Additionally, vasomotor control can be regulated by HIF, and so activation of HIF might lower systemic blood pressure.

By “treatment”, is meant any degree of alleviation of the disease, including slowing its development. This will be beneficial in increasing the time until alternative treatment (such as surgery) is required. Treatment is also intended to include prophylaxis, e.g., to prevent ischaemia, for example in the promotion of coronary collaterals in the treatment of angina.

In the present invention these diseases may be treated by the administration of a modulator of VDU1.

By “modulator” of a protein (e.g., VDU1), is meant an inhibitor or activator, i.e., an agent which reduces or enhances the total activity of the protein.

By “inhibitor” is meant an agent which reduces the total activity of the protein. This may be by reducing the total amount of protein in the cell, preferably by reducing the expression of that protein. Alternatively, it may be inhibition which occurs by reducing the ability of a protein to perform its function.

Similarly, by “activator” is meant an agent which increases total protein activity. This may be by increasing the total amount of protein in the cell, preferably by enhancing expression, or by increasing the ability of the protein to perform its function.

The terms “modulation”, “inhibition” and “activation” are to be interpreted accordingly.

Preferably, a modulator of VDU1 is a specific modulator, that is, one which directly affects the protein activity of VDU1 but does not directly affect the stability of HIF-α. More preferably, it does not directly affect the activity of any cellular protein other than VDU1. In particular, it is preferred that the modulator of VDU1 is not VHL, as VHL directly targets HIF-α for degradation as well as VDU1 (Li et al, 2002).

In some embodiments a modulator may be obtainable or obtained by an assay method of the invention, as described above.

An inhibitor or activator of VDU1 may be a natural or synthetic chemical compound.

Modulators most suited for therapeutic applications will be small molecules e.g. selected from a combinatorial library such as are now well known in the art (see e.g. Newton (1997) Expert Opinion Therapeutic Patents, 7(10): 1183-1194). Candidate substances may include small molecules such as those of the steroid, benzodiazepine or opiate classes.

Another class of modulator is polypeptides. An example of an inhibitor polypeptide is a polypeptide derived from the VHL or VDU1 protein sequences, which inhibits the interaction between these two proteins. The peptide fragments may be fragments of from 5 to 40 amino acids, for example from 6 to 10 amino acids from the regions of VHL or VDU1 which are responsible for the interaction between these proteins.

Other possible modulator polypeptides are anti-VDU1 agonist or antagonist antibodies. Candidate modulator antibodies may be characterised and their binding regions determined to provide single chain antibodies and fragments thereof which are responsible for modulating VDU1 activity. Antibodies may be human, or humanised.

A VDU1 antibody is specific, in the sense of being able to distinguish between the polypeptide it is able to bind and other polypeptides of the same species for which it has no or substantially no binding affinity (e.g. a binding affinity of at least about 1000× worse). Specific antibodies bind an epitope on the molecule which is either not present or is not accessible on other molecules.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies may be obtained using techniques which are standard in the art. Methods of producing antibodies include immunising a mammal (e.g. mouse, rat, rabbit) with a polypeptide of the invention. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992).

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, C1 and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

A monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-0239400. Cloning and expression of chimeric antibodies are described in EP-A-0120694 and EP-A-0125023.

Preferred activators of VDU1 activity are agents which enhance the expression of VDU1. Such agents may for example be those which enhance the production of native VDU1 in the cell, or they may be a nucleic acid which encodes VDU1, for example a gene therapy vector designed to express VDU1 in target cells.

An inhibitor may be a nucleic acid comprising a sequence corresponding to or complementary to all or part of the sequence of a VDU1 nucleic acid molecule, such that when the modulator is present in a cell VDU1 expression is reduced. Such inhibitors may for example be anti-sense RNA, siRNA or a double-stranded RNA which will be processed in the cell to form siRNA, as explained below. Other possible nucleic acid inhibitors include ribozymes which target VDU1 mRNA. These agents may be directed to VDU mRNA in target cells in the individual, in order to reduce expression of the gene. The nucleic acids may be delivered as naked nucleic acid or formulations thereof, e.g., liposomal formulations designed to enhance cellular uptake. DNA molecules or gene therapy vectors which express the nucleic acid in the target cell may also be used. Examples of suitable vectors are discussed further below.

RNA interference is a two step process. First, dsRNA is cleaved within the cell to yield short interfering RNAs (siRNAs) of about 21-23 nt length with 5′ terminal phosphate and 3‘short overhangs’ (˜2 nt) The siRNAs target the corresponding mRNA sequence specifically for destruction (Zamore P. D. Nature Structural Biology, 8, 9, 746-750, (2001) Thus, the siRNA inhibitor may be a double stranded RNA comprising a VDU1-encoding sequence, which may for example be a “long” double stranded RNA (which will be processed to siRNA, e.g., as described above). These RNA products may be synthesised in vitro, e.g., by conventional chemical synthesis methods.

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

Thus a VDU1 inhibitor may also be a siRNA duplex containing between 20 and 25 bps, more preferably between 21 and 23 bps, of the VDU1 sequence, e.g. as produced synthetically, optionally in protected form to prevent degradation. Alternatively siRNA may be produced from a vector, in vitro (for recovery and use) or in vivo.

In one embodiment, the vector may comprise a nucleic acid sequence corresponding to part of the VDU1 sequence in both the sense and antisense orientation, such that when expressed as RNA the sense and antisense sections will associate to form a double stranded RNA. This may for example be a long double stranded RNA (e.g., more than 23 nts) which may be processed in the cell to produce siRNAs (see for example Myers (2003) Nature Biotechnology 21:324-328). Alternatively, the double stranded RNA may directly encode the sequences which form the siRNA duplex. In another embodiment, the sense and antisense sequences are provided on different vectors.

A ribozyme is a catalytic RNA molecule that cleaves other RNA molecules having particular nucleic acid sequences. General methods for the construction of ribozymes, including hairpin ribozymes, hammerhead ribozymes, RNAse P ribozymes (i.e., ribozymes derived from the naturally occurring RNAse P ribozyme from prokaryotes or eukaryotes) are known in the art. Castanotto et al (1994) Advances in Pharmacology 25: 289-317 provides an overview of ribozymes in general, including group I ribozymes, hammerhead ribozymes, hairpin ribozymes, RNAse P, and axhead ribozymes.

Agents which modulate VDU1 may be administered to a subject in need of treatment in any suitable form. Usually the agent will be in a form of a pharmaceutical composition in which the agent is mixed with a pharmaceutically acceptable carrier. The carrier will be adapted to be suitable for the desired route of administration of the agent. The agent may be administered, for example, orally, topically, subcutaneously or by other routes.

In general, pharmaceutical compositions contemplated for use in the present invention can be in the form of a solid, a solution, an emulsion, a dispersion, a micelle, a liposome, and the like, wherein the resulting composition contains one or more of the active compounds contemplated for use herein, as active ingredients thereof, in admixture with an organic or inorganic carrier or excipient suitable for enteral or parenteral applications. The active ingredients may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form.

Pharmaceutical compositions containing the active ingredients contemplated herein may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions.

In some cases, formulations for oral use may be in the form of hard gelatin capsules wherein the active ingredients are mixed with an inert solid diluent, for example, calcium carbonate, calcium phosphate, kaolin, or the like. They may also be in the form of soft gelatin capsules wherein the active ingredients are mixed with water or an oil medium, for example, peanut oil, liquid paraffin, or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

For example, formulations of compounds for topical administration include transdermal formulations designed to enhance uptake of the active agent through the skin. Transdermal delivery devices, e.g. patches, are well known in the art and may be used to present a transdermal formulation of the agent.

The amount of agent administered will be dependent upon the nature of the agent and its route and dose of administration, whilst also taking into account the patient and their particular needs.

Gene therapy of somatic cells can be accomplished by using, e.g., retroviral vectors, other viral vectors, or by non-viral gene transfer (for clarity cf. T. Friedmann, Science 244 (1989) 1275; Morgan 1993, RAC DATA MANAGEMENT REPORT, June 1993).

Vector systems suitable for gene-therapy are, for instance, retroviruses (Mulligan, R. C. (1991) in Nobel Symposium 8: Ethiology of human disease at the DNA level (Lindsten, J. and Pattersun Editors), pages 143-189, Raven Press), adeno associated virus (McLughlin, J. Virol. 62 (1988), 1963), vaccinia virus (Moss et al., Ann. Rev. Immunol. 5 (1987) 305), bovine papilloma virus (Rasmussen et al., Methods Enzymol. 139 (1987) 642) or viruses from the group of the herpes viruses such as Epstein Barr virus (Margolskee et al., Mol. Cell. Biol. 8 (1988) 2937) or Herpes simplex virus.

There are also known non-viral delivery systems. See for example U.S. Pat. No. 6,228,844 (Wolff). For this, usually “nude” nucleic acid, preferably DNA, is used, or nucleic acid together with an auxiliary such as, e.g., transfer reagents (liposomes, dendromers, polylysine-transferrine-conjugates (Wagner, 1990; Felgner et al., Proc. Natl. Acad. Sci. USA 84 (1987) 7413)).

Gene therapy vectors comprising a sequence encoding VDU1 operably linked to a promoter functional in the target cells may thus be used to stabilise HIF-α and to increase HIF-mediated responses.

In another embodiment, the gene therapy vector may comprise a sequence which corresponds to or is complementary to all or part of the VDU1 sequence operably linked to a promotor, which may be used to decrease the expression of VDU1 and to de-stabilise HIF-α, reducing HIF-mediated responses, as discussed above.

Promoters suitable for use in various vertebrate systems are well known. For example, strong promoters include RSV LTR, MPSV LTR, SV40 IEP, and metallothionein promoter. The CMV IEP may be more preferable for human use.

CYLD

As indicated above, individuals with cylindromatosis are those with a lesion in the CYLD gene located on chromosome 16q12-13 leading to a mutation or lack of expression of the CYLD gene product. Bignell et al, ibid, report the identification of the structure of CYLD and report that many affected individuals have mutations located in the 3′ two-thirds of the CYLD coding sequence. Other human individuals may have deletions of the entire region of the chromosome where the gene is located.

Individuals with cylindromatosis can be administered an effective amount of an NF-κB inhibitor for the treatment of their condition.

By“treatment”, it is meant any degree of alleviation of the disease including a suppression in the rate of growth of the tumours. This will be beneficial in lengthening the time before surgical intervention is required.

A number of agents which are known to inhibit NF-κB are known in the art. For example, U.S. Pat. No. 5,985,592 discloses that pentoxifylline or functional derivatives or metabolites thereof can be used for the treatment of diseases characterised by activation of NF-κB. The phrase “pentoxifylline or functional derivatives/metabolites thereof” refers to the compound 1-(5-oxohexyl)-3,7-dimethylxanthine (pentoxifylline), and oxidation-, reduction-, substitution- and/or rearrangement-products thereof, such as, for example, metabolite-1 through metabolite-7 as described by Luke and Rocci in J. Chromatogr.374(1):191-195 (1986) (e.g., 1-(5-hydroxyhexyl)-3,7-dimethyl-xanthine (metabolite-1)), as well as synthetic variants thereof (e.g., propentofylline).

U.S. Pat. No. 6,090,542 teaches that NF-κB activity may be suppressed by treating cells with a substance which inhibits the proteolytic degradation of the alpha subunit of IκB, IκB-α.

Other agents which are known to inhibit NF-κB include aspirin, ibuprofen, sulindac, flurbiprofen and salicylates; and cyclopentenone prostaglandins (cyPGs) such as A-type and J-type cyPGs, for example prostaglandin A1 (PGA1) and cyPG 15-deoxy-delta12-14-PGJ2.

A further class of agents comprises nucleic acids including anti-sense nucleic acids, siRNA and ribozymes. These agents may be directed to NF-κB mRNA in target cells in the individual, in order to reduce expression of the gene. The nucleic acids may be delivered as naked DNA or formulations thereof, e.g. liposomal formulations designed to enhance cellular uptake. Gene therapy vectors which express the nucleic acids in the target cells may also be used.

Agents which inhibit NF-κB may be administered to a subject in need of treatment in any suitable form. Usually the agent will be in a form of a pharmaceutical composition in which the agent is mixed with a pharmaceutically acceptable carrier. The carrier will be adapted to be suitable for the desired route of administration of the agent. The agent may be administered, for example, orally, topically, subcutaneously or by other routes.

In general, pharmaceutical compositions contemplated for use in the present invention can be in the form of a solid, a solution, an emulsion, a dispersion, a micelle, a liposome, and the like, wherein the resulting composition contains one or more of the active compounds contemplated for use herein, as active ingredients thereof, in admixture with an organic or inorganic carrier or excipient suitable for enteral or parenteral applications. The active ingredients may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form.

Pharmaceutical compositions containing the active ingredients contemplated herein may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions.

In some cases, formulations for oral use may be in the form of hard gelatin capsules wherein the active ingredients are mixed with an inert solid diluent, for example, calcium carbonate, calcium phosphate, kaolin, or the like. They may also be in the form of soft gelatin capsules wherein the active ingredients are mixed with water or an oil medium, for example, peanut oil, liquid paraffin, or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

For example, formulations of compounds for topical administration include transdermal formulations designed to enhance uptake of the active agent through the skin.

Transdermal delivery devices, e.g. patches, are well known in the art and may be used to present a transdermal formulation of the agent. For example, U.S. Pat. No. 6,368,618 describes a formulation suitable for the transdermal administration of aspirin comprising aspirin (e.g. in an amount 1% to about 30% w/w) together with at least one alcohol (e.g. in an amount 1% to about 40% w/w) selected from the group consisting of isopropyl alcohol, ethyl alcohol and propylene glycol; and at least one melting point depressing agent selected from the group consisting of thymol, menthol, eucalyptol, eugenol, methyl salicylate, phenyl salicylate, capsaicin, butylated hydroxytoluene, a local anesthetic agent and any combination thereof, said melting point depressing agent present in the composition in an amount of less than about ¼ (e.g. from 1/20 to ¼) of the weight of the aspirin; said composition having spontaneously equilibrated aqueous and oil phases, wherein the aspirin is in substantially melted form at 25° C., and wherein the concentration of the aspirin in the oil phase is, by weight, at least about 40% of the weight of the oil phase.

The amount of agent administered will be dependent upon the nature of the agent and its route and dose of administration, and taking into account the patient and their particular needs. For example, aspirin and other NSAIDs administered orally can be provided in a unit dosage form of from 100 to 1000 mg, to be taken 1 to 5 times a day. Other routes of administration of the same drug may be dosed to an equivalent level. Reference may be made to U.S. Pat. No. 5,985,592 for doses of pentoxifylline or functional derivatives or metabolites thereof. Prostaglandins may be administered in the range of 0.1 to 100 mg/kg body weight per day.

It is possible that CYLD may also be mutated in other cancers, such as breast, lung, colon and prostate cancers. Thus the agents and their formulations and routes and doses of delivery referred to herein may be used in the treatment of other cancer conditions associated with a mutation in the CYLD gene.

The role of CYLD in suppressing the anti-apoptopic effects of NF-κB provides the potential to treat diseases associated with cellular proliferation by enhancing the levels of CYLD in a cell in order to suppress the release of NF-κB from IκB. Such diseases include interstitial lung disease, human fibrotic lung disease (e.g., idiopathic pulmonary fibrosis (IPF), adult respiratory distress syndrome (ARDS), tumor stroma in lung disease, systemic sclerosis, Hermansky-Pudlak syndrome (HPS), coal worker's pneumoconiosis (CWP), chronic pulmonary hypertension, AIDS associated pulmonary hypertension, and the like), human kidney disease (e.g., nephrotic syndrome, Alport's syndrome, HIV-associated nephropathy, polycystic kidney disease, Fabry's disease, diabetic nephropathy, and the like), glomerular nephritis, nephritis associated with systemic lupus, liver fibrosis, myocardial fibrosis, pulmonary fibrosis, Grave's ophthalmopathy, drug induced ergotism, cardiovascular disease, cancer, Alzheimer's disease, scarring, scleroderma, glioblastoma in Li-Fraumeni syndrome, sporadic glioblastoma, myeloid leukemia, acute myelogenous leukemia, myelodysplastic syndrome, myeloproliferative syndrome, cancers such as breast, lung, colon, prostate or gynecological cancer (e.g., ovarian cancer, Lynch syndrome, and the like), Kaposi's sarcoma, Hansen's disease, inflammatory bowel disease, and the like.

Elevating the levels of CYLD may be achieved by administration of an agent which enhances the production of native CYLD in the cell, or by introduction of a gene therapy vector designed to express CYLD in target cells. Gene therapy of somatic cells can be accomplished by using, e.g., retroviral vectors, other viral vectors, or by non-viral gene transfer (for clarity cf. T. Friedmann, Science 244 (1989) 1275; Morgan 1993, RAC DATA MANAGEMENT REPORT, June 1993).

Vector systems suitable for gene therapy are, for instance, retroviruses (Mulligan, R. C. (1991) in Nobel Symposium 8: Ethiology of human disease at the DNA level (Lindsten, J. and Pattersun Editors), pages 143-189, Raven Press), adeno associated virus (McLughlin, J. Virol. 62 (1988), 1963), vaccinia virus (Moss et al., Ann. Rev. Immunol. 5 (1987) 305), bovine papilloma virus (Rasmussen et al., Methods Enzymol. 139 (1987) 642) or viruses from the group of the herpes viruses such as Epstein Barr virus (Margolskee et al., Mol. Cell. Biol. 8 (1988) 2937) or Herpes simplex virus.

There are also known non-viral delivery systems. See for example U.S. Pat. No. 6,228,844 (Wolff). For this, usually “nude” nucleic acid, preferably DNA, is used, or nucleic acid together with an auxiliary such as, e.g., transfer reagents (liposomes, dendromers, polylysine-transferrine-conjugates (Wagner, 1990; Felgner et al., Proc. Natl. Acad. Sci. USA 84 (1987) 7413)).

Gene therapy vectors comprising a sequence encoding CYLD (the sequence of which is available in Bignell et al, ibid, operably linked to a promoter functional in the target cells may thus be used to suppress the anti-apoptopic effects of NF-κB. Promoters suitable for use in various vertebrate systems are well known. For example, strong promoters include RSV LTR, MPSV LTR, SV40 IEP, and metallothionein promoter. The CMV IEP may be more preferable for human use.

Assays according to the invention may be performed in any cell line, preferably a mammalian cell line, more preferably a human cell line, in which the NF-κB pathway is active. The cells will either naturally contain deficient CYLD (e.g. by originating from a subject with cylindromatosis) or may be modified to suppress, either temporally or permanently, the CYLD gene. Suppression of the activity of CYLD may be achieved by siRNA, as illustrated in the accompanying examples.

In assays of the invention, a cell culture in which CYLD activity is suppressed or missing will be brought into contact with an agent to be assayed. Following incubation of the cells, for example from 1 to 48 hours, the activity of NF-κB will be determined.

The amount of an agent which may be added to an assay of the invention will normally be determined by trial and error depending upon the type of compound used. Typically, from about 0.01 to 100 nM concentrations of agent compounds may be used, for example from 0.1 to 10 nM. Agent compounds which may be used may be natural or synthetic chemical compounds used in drug screening programmes. Extracts of plants or microorganisms which contain several characterised or uncharacterised components may also be used.

The activity of NF-κB may be determined by a variety of means. For example, the amount of NF-κB protein in a cell may be examined by immunological techniques, such as western blotting. The amount of NF-κB RNA in the cell may be examined, using for example northern blotting or quantitative PCR. Alternatively, the amount of NF-κB can be examined using a reporter gene assay, i.e. by determining the amount of expression of a reporter gene (e.g. firefly luciferase, secreted alkaline phosphatase (SEAP) or green fluorescent protein) whose promoter comprises one or more (e.g. two, three or four) tandem copies of the k enhancer element Such constructs are commercially available (e.g. the pNF-κB-Luc vector from BD Biosciences Clontech, Palo Alto, Calif.).

The following examples illustrate the invention.

### EXAMPLES

Protein ubiquitination is used primarily to target proteins for proteasome-mediated destruction¹. Protein ubiquitination is a dynamic process that involves large families of ubiquitin-conjugating enzymes and ubiquitin ligases that add ubiquitin molecules to substrates and a less-studied family of deubiquitinating enzymes (DUBs) that remove ubiquitin from protein substrates. Two classes of DUBs can be distinguished: ubiquitin C-terminal hydrolases (UCHs) and ubiquitin-specific processing proteases (UBPs)^{1,3}. The UBP enzymes remove ubiquitin residues from large substrates by cleaving at the C-terminus of the ubiquitin moiety and are candidate antagonists of the ubiquitin conjugation/ligation system. A role for DUB genes in cancer is suggested by the fact that this family contains both oncogenes^{6,7} and tumour suppressor genes⁴. In addition, members of the DUB family have been described to interact with p53⁸ and BRCA1⁹ and the von Hippel Lindau (VHL) tumour suppressor gene¹⁰.

### Example 1

The strategy we pursued to study the function of the individual-members of this family of DUB enzymes was to inhibit the expression of independent family members through RNA interference and search for phenotypes induced by loss of DUB expression. We first searched several nucleotide sequence databases for genes with homology to the catalytic domain of DUBs. A total of 50 genes could be identified harbouring this motif, including the cylindromatosis tumour suppressor gene (CYLD)⁴ and the TRE2 oncogene⁶, and DUB no. 33, which corresponds to VDU1.

Next, we retrieved the cDNA sequences corresponding to these potential DUBs and selected four unique 19-mer sequences from each transcript for cloning into PSUPER, a vector that mediates suppression of gene expression through the synthesis of short hairpin RNAs having siRNA-like properties¹¹. We chose to make four knockdown vectors against each DUB to increase the chance that a significant inhibition of DUB expression would be obtained. In total, we made 200 knockdown vectors, which were subsequently pooled into 50 sets of 4 vectors, where each set of vectors was designed to target a single DUB transcript.

To ask how effective the set of four knockdown vectors inhibited DUB gene expression, we fused the open reading frame of four of the DUBs to GFP and determined the levels of GFP-DUB fusion protein expression in the absence and presence of co-expression of the DUB knockdown vectors. 293 cells were co-transfected and immunoblotted with a GFP antibody. P21-RFPserved as a transfection control. A significant reduction in protein levels was induced by all four DUB knockdown vectors, whereas control p21-RFP fusion protein was unaffected. We conclude that this strategy allows efficient inhibition of DUB expression.

### Example 2

The four pSUPER VDU1 knockdown vectors contained the following sequences:

SEQ ID NO:1

GATCCCCGAGCCAGTCGGATGTAGATTTCAAGAGAATCTACATCCGACTG
GCTCTTTTTGGAAA

SEQ ID NO:2

GATCCCCGTAAATTCTGAAGGCGAATTTCAAGAGAATTCGCCTTCAGAAT
TTACTTTTTGGAAA

SEQ ID NO:3

GATCCCCGCCCTCCTAAATCAGGCAATTCAAGAGATTGCCTGATTTAGGA
GGGCTTTTTGGAAA

SEQ ID NO:4

GATCCCCGTTGAGAAATGGAGTGAAGTTCAAGAGACTTCACTCCATTTCT
CAACTTTTTGGAAA

Each of the four sequences contains sense and antisense sequence, and form hairpin loops when expressed in cells. These hairpins are converted by the cell into double-stranded siRNA molecules.

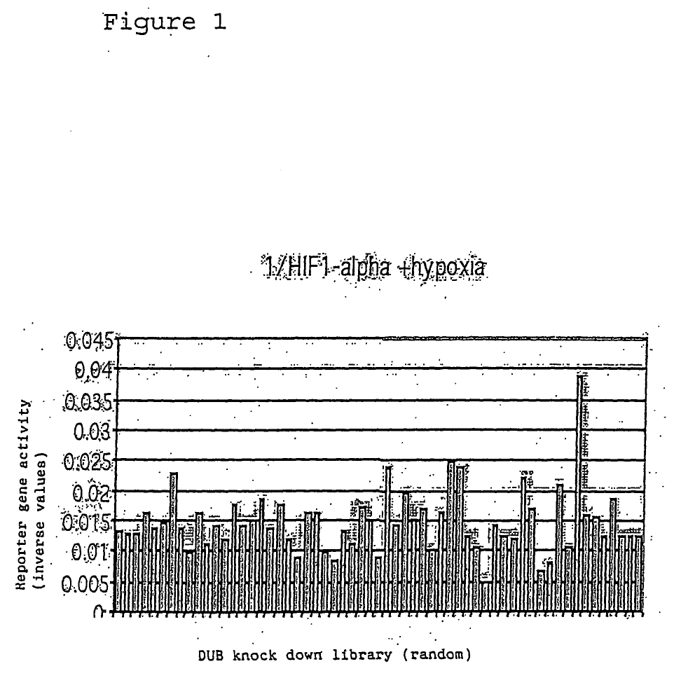

To identify the function individual members of the DUB family, we used a hypoxia inducible reporter (3×RE-luciferase HIFα responsive reporter) to measure the effect of the loss of DUB expression. Under normoxic conditions HIF-1α is a protein with a very short half-life due to continued VHL mediated ubiquitination. Under hypoxic conditions however HIF-1α is rapidly stabilized and functions as a transcriptional activator to stimulate transcription of target genes. FIG. 1 shows that loss of DUB no. 33 in HEP-G2 cells results in decreased activity of the hypoxia inducible reporter under hypoxic conditions (the graph shows inverse values).

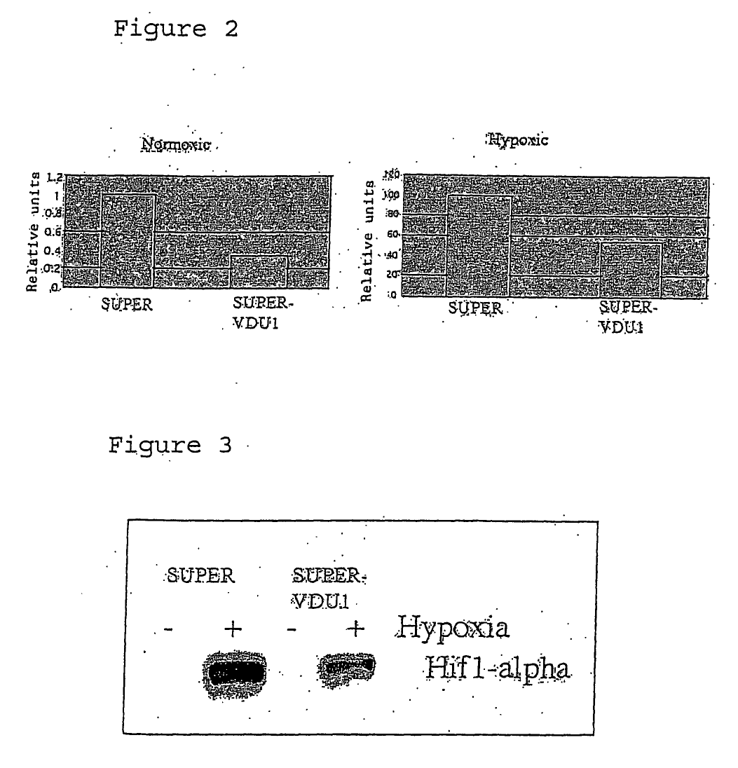

As indicated in FIG. 2 VDU-1 knock-down results in a decrease of reporter activity under both hypoxic and normoxic conditions. SV40 Renilla luciferase served as an internal control.

To examine whether loss of VDU-1 not only affects reporter gene activity but also truly affects the hypoxia response in cells we transfected U2OS cells with pSUPER-VDU-1 and pSUPER control vectors and monitored HIF-1α activation by western blot. As expected 12 hrs treatment of cells with the hypoxia mimetic desferrioxiamine results in increased HIF-1α, whereas cells transfected with pSUPER-VDU-1 show a marked decrease of HIF-1α levels under hypoxic conditions (FIG. 3). As HIF-1α is mainly regulated by ubiquitin induced protein degradation and loss of the deubiquinating enzyme VDU-1 affects HIF-1α protein levels, it is very likely that VDU-1 acts on HIF-1α to remove attached ubiquitin chains. This would suggest that full activation of HIF-1α could only be obtained under conditions where ubiquitination is inhibited and HIF-1α is subjected to active deubiquitination.

Materials and Methods

Materials, Antibodies, and Plasmids Construction.

To generate DUB knockdown vectors, four annealed sets of oligonucleotides encoding short hairpin transcripts corresponding to one DUB enzyme were cloned individually into pSUPER. Bacterial colonies were pooled and used for plasmid preparation. To generate GFP-DUB fusion proteins the corresponding DUB enzymes were PCR amplified using DNA from human cDNA libraries as a template and cloned into pEGFP-N1. The hypoxia inducible reporter plasmid was a 3× hypoxia responsive element linked to luciferase.

Cell Cultures, Transient Transfections and Reporter Assays.

All cells were cultured in Dulbecco's modified Eagle medium (DMEM) supplemented with 10% fetal calf serum. High efficiency electroporation of cells was done as described²⁰. Reporter assays were carried out using calcium-phosphate transfection of 0.5 μg 3×RE-luciferase, 1 ng SV40-Renilla and 2.5 μg pSUPER vectors. To mimic hypoxia, cells were exposed to 12 hrs 1 mM Desferrioxamine (DFO) starting 48 hours after transfection.

Immunoblotting

Western blots were performed using whole cell extracts, separated on 8-12% SDS-PAGE gels and transferred to polyvinylidine difluoride membranes (Millipore). Western blots were probed with the indicated antibodies. Transformed human embryonic kidney cells (293 cells) were transfected by calcium-phosphate precipitation with the indicated plasmids, 48 hrs post-transfection cells were lysed in ELB buffer (0.25M NaCL, 0.1% NP-40, 50 mM Hepes pH 7.3) supplemented with “Complete” protease inhibitors (Roche), centrifuged and protein complexes were immunoprecipitated with 2 μg of the indicated antibodies conjugated to protein G sepharose beads. The beads were washed four times with ELB buffer and protein complexes were eluted by boiling in SDS-sample buffer and resolved on 10% SDS-PAGE.

### Example 3

To further study the function of the members of the DUB family, we asked if suppression of any of the DUBs could affect the activity of NF-κB, a cancer-relevant transcription factor with marked anti apoptotic activity¹². We transfected an NF-κB-luciferase reporter gene (3×RE), together with each of the 50 sets of 4 DUB knockdown vectors into human U2-OS cells and after 48 hours measured the effect of DUB knockdown on Tumour Necrosis Factor-α (TNF-α)-activated levels of NF-κB by stimulation overnight with TNF-α (20 ng/ml) and measurement of luciferase activity. SV40 Renilla luciferase served as an internal control.

The four pSUPER CYLD (DUB36) knockdown vectors contained the following sequences:

SEQ ID NO:5

GATCCCCCAGTTATATTCTGTGATGTTTCAAGAGAACATCACAGAATATA
ACTGTTTTTGGAAA

SEQ ID NO:6

GATCCCCGAGGTGTTGGGGACAAAGGTTCAAGAGACCTTTGTCCCCAACA
CCTCTTTTTGGAAA

SEQ ID NO:7

GATCCCGTGGGCTCATTGGCTGAAGTTCAAGAGACTTCAGCCAATGAGCC
CACTTTTTGGAAA

SEQ ID NO:8

GATCCCCGAGCTACTGAGGACAGAAATTCAAGAGATTTCTGTCCTCAGTA
GCTCTTTTTGGAAA

Each of the four sequences contains sense and antisense sequence, and form hairpin loops when expressed in cells. These hairpins are converted by the cell into double-stranded siRNA molecules.

Only one of the sets of DUB knockdown vectors (#36) significantly enhanced TNF-α-activation of NF-κB. This effect was specific, as knockdown of DUB#36 did not affect an E2F-luciferase reporter or a Hypoxia Induced Factor 1-α (HIF-1α)-responsive promoter (data not shown). Importantly, the DUB#36 set of knockdown vectors targets the cylindromatosis tumour suppressor gene CYLD⁴, a confirmed de-ubiquitinating enzyme¹³, suggesting that CYLD is a regulator of NF-κB.

To ask if the CYLD knockdown vectors efficiently suppress abundance of the CYLD protein, we generated an HA-epitope-tagged CYLD expression vector and co-transfected this vector with the pSUPER-CYLD knockdown vector, the most active of the four CYLD knockdown vectors in the initial pool of four CYLD knockdown vectors. U2-OS cells were transfected with HA-tagged CYLD and pSUPER-CYLD or empty vector. Whole cell extracts were immunoblotted with an HA antibody. GFP served as a transfection control. HA-CYLD protein levels were significantly reduced by pSUPER-CYLD, confirming that CYLD is efficiently targeted for suppression by the CYLD knockdown vector.

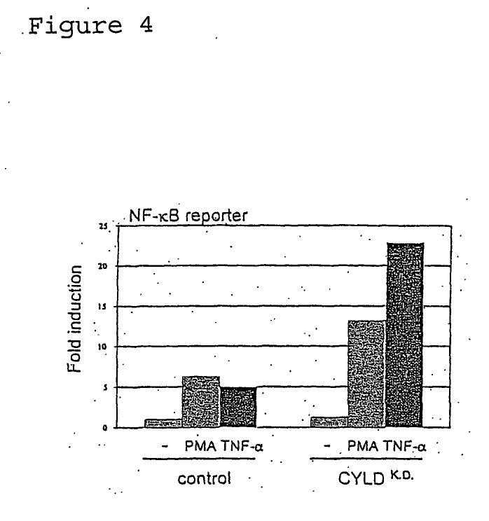

NF-κB is held in an inactive form in the cytoplasm by IκB inhibitor proteins. Signalling through the IκB kinase (IKK) complex, containing the IκB kinases IKKα and β and the structural component NEMO (or IKKγ), causes phosphorylation and subsequent degradation of IκB, allowing nuclear translocation of NF-κB^{12,14}. In principle, the observed effect of CYLD knockdown on TNF-α stimulation of NF-κB could result from an effect of CYLD on the TNF-α receptor, a more downstream effect on the IKK complex or directly on the IκB/NF-κB complex itself. Since the tumour promoter phorbol 12-myristate 13-acetate (PMA) activates NF-κB downstream of the TNF-α receptor, we asked if CYLD knockdown also affected PMA-mediated activation of NF-κB. FIG. 4 shows that CYLD knockdown did not enhance basal level of NF-κB activity, but further increased both PMA and TNF-α activated NF-κB levels. This suggests that CYLD loss affects NF-κB downstream of the TNFα receptor.

Next, we asked if CYLD could physically associate with known members of the NF-κB signalling machinery. In this experiment, 293 cells were transfected as indicated, lysates were prepared 48 hours later and the protein complexes immunoprecipitated (IP) using Flag antibody. Ips were immunoblotted for HA-tagged CYLD and whole cell extracts were immunoblotted fro Flag-tagged IκBα, IκKβ and NEMO/IKKγ and for HA-tagged CYLD. CYLD co-immunoprecipitated specifically with NEMO/IKKγ, but not with IκBa or IκBα. This suggests that CYLD acts on the IκB kinase complex through direct association. To address this, we measured IKKβ kinase activity following TNF-α stimulation in the presence and absence of CYLD knockdown, using an in vitro kinase assay. In summary, U2-S cells were co-transfected with Flag-tagged IKKβ and pSUPER-CYLD or empty vector. Cells were stimulated as indicated, IKKβ was immunoprecipitated from cell lysates and incubated with GST-IκBα (1-72) in the presence of ³²P-γATP. Immunoprecipitated IKKβ was visualised by immunoblotting with Flag antibody. In the absence of TNF-α, no IKKβ kinase activity towards IκBα could be detected. As expected, TNF-α treatment significantly stimulated IKKβ kinase activity. Importantly, this activity was further enhanced when cells were co-transfected with CYLD knockdown vector. No effects were seen of CYLD knockdown on IKKβ protein levels suggesting that CYLD does not act to regulate IKKβ abundance.

Consistent with an increase in IKKβ kinase activity by CYLD knockdown, we observed that CYLD knockdown resulted in a more significant reduction in IκBα levels, an endogenous substrate of IKKβ kinase. To test this, U2-OS cells were electroporated with pSUPER-CYLD or empty vector, together with a puromycin resistance marker. Transfected cells were selected for 48 hours with puromycin (2.0 μg/ml) and stimulated with TNF-α (15 ng/ml). Whole cell extracts were immunoblotted for endogenous IκBα. Together, these data indicate that CYLD acts as an antagonist of the IKK complex through direct binding to the non-catalytic NEMO/IKKγ component and that reduction of CYLD expression stimulates signalling through the IKK complex.

When combined with inhibitors of transcription or translation, TNF-α is a potent inducer of apoptosis in certain cell types.

This pro-apoptotic activity of TNF-α can be inhibited by simultaneous activation of NF-κB; which activates a number of anti-apoptotic genes¹⁵. Since CYLD knockdown stimulates PMA-induced activation of NF-κB, we asked if CYLD and PMA also collaborate to inhibit TNF-α induced apoptosis. To address this, we treated Hela cells with TNF-α in the presence of cycloheximide (CHX) to induce apoptosis both with and without pre-treatment with PMA (see methods). 12-hour treatment with TNF-α efficiently induced apoptosis in some 95% of the Hela cells. As expected, pre-treatment with PMA resulted in an approximately four-fold increase of the number of viable cells. Significantly, PMA pre-treatment in Hela cells that had been transfected with CYLD knockdown vector resulted in an even larger fraction of surviving cells, suggesting that loss of the CYLD tumour suppressor gene confers resistance to induction of apoptosis, most likely through activation of NF-κB. Consistent with this notion, CYLD knockdown and PMA also collaborated in NF-κB activation in Hela cells.

NF-κB can be inhibited by a number of pharmacological agents, including aspirin and prostaglandin A1 (PGA1)^{5,16}. Both compounds have been shown to act on IKKβ, the same kinase that is hyper-activated as a result of CYLD knockdown. This raises the possibility that the effect of CYLD knockdown on NF-κB activation can be counteracted by aspirin or PGA1. To address this, we transfected U2-OS cells with the NF-κB-luciferase reporter plasmid and activated NF-κB 48 hours after transfection with PMA (200 nM) or PMA and aspirin (10 mM) or PMA and prostaglaridin A1 (8 μM). As was observed before, knockdown of CYLD further enhanced PMA-stimulated activation of NF-κB. Strikingly, this effect of CYLD knockdown on NF-κB activity could be significantly suppressed both by aspirin and by PGA1, indicating that these compounds can compensate for CYLD suppression in this assay.

As was discussed above, it is possible that loss of the CYLD tumour suppressor gene confers resistance to apoptosis through activation of NF-κB. If this notion is correct, one would expect that the protective effect of CYLD knockdown on apoptosis can be reversed by the NF-κB inhibitor aspirin. We tested this by treating Hela cells with TNF-α (10 ng/ml) and PMA (200 ng/ml) or PMA and aspirin (8 mM) in the presence of CYLD knockdown. Cycloheximide (10 μg/ml) was used alongside TNF to induce apoptosis. The combination of CYLD knockdown and PMA treatment again conferred significant resistance to TNF-α-induced apoptosis. Significantly, exposure of cells to 10 mM aspirin prior to TNF-α treatment completely abolished the protective effect of CYLD knockdown on TNF-α-induced apoptosis, indicating that aspirin can also reverse the anti-apoptotic effects of CYLD loss. This result is consistent with the notion that CYLD knockdown protects from TNF-α-induced apoptosis through activation of IKKβ and subsequently of NF-κB.

We describe here the first high-throughput RNA interference screen in mammalian cells to identify novel regulators of NF-κB. We focused on ubiquitin-specific processing proteases (UBPs) as these proteins are potential antagonists of the well-studied ubiquitin conjugating enzymes and ubiquitin ligases. Unexpectedly, we identify the familial cylindromatosis tumour suppressor gene (CYLD) as a novel negative regulator of NF-κB, thus establishing the first direct link between the NF-κB signalling cascade and a tumour suppressor gene. Our results provide an explanation for the deregulated proliferation of the epidermal appendices in patients with familial mutations in the CYLD gene. It is well-established that NF-κB is required for normal skin proliferation¹⁴. For instance, mice with suppressed NF-κB have defects in the development of hair follicles and exocrine glands, resulting from increased rates of apoptosis¹⁷. Furthermore, female NEMO/IKKγ heterozygous mice have severe skin defects, including increased apoptosis in keratinocytes¹⁸. A similar skin defect is found in the human genetic disorder incontinentia pigmenti (IP), which also results from mutations in the NEMO/IKKγ gene¹⁹. Thus, inhibition of NF-κB in the skin causes an increase in apoptosis. We therefore suggest that the deregulated proliferation in the skin appendices in patients suffering from cylindromatosis results from a perturbation in the balance between proliferation and apoptosis in favor of proliferation, resulting from an increase in active NF-κB. That cylindromas result from a relatively mild perturbation of normal proliferation is also supported by the notion that most cylindromas have a diploid karyotype and are rarely metastatic⁴. The observation that the enhanced protection from apoptosis that results from CYLD suppression can be reversed by simple pharmacological agents like aspirin and prostaglandin A1 suggests a strategy to restore normal growth control in patients suffering from familial cylindromatosis.

Methods.

Materials, Antibodies, and Plasmids Construction.

To generate DUB knockdown vectors, four annealed sets of oligonucleotides encoding short hairpin transcripts corresponding to one DUB enzyme were cloned individually into pSUPER. Bacterial colonies were pooled and used for plasmid preparation. To generate GFP-DUB fusion proteins the corresponding DUB enzymes were PCR amplified using DNA from human cDNA libraries as a template and cloned into pEGFP-N1. pNF-κB-Luc vector was obtained from Clontech, SV40-Renilla from Promega. PMA, TNF-α and Prostaglandin A1 and cycloheximide were purchased from Sigma. HA-tagged CYLD was PCR amplified from human cDNA libraries and cloned into pcDNA 3.1 (−), Flag tagged NEMO was generated by cloning an EcoRI-XbaI NEMO containing fragment into pcDNA-flag. Anti-IκB-α (c-21) and HA tag (Y-11) antibodies were obtained from Santa Cruz, anti-Flag M2 from Sigma and anti-GFP rabbit polyclonal serum was kindly provided by J. Neefjes.

Cell Cultures, Transient Transfections and Reporter Assays.

All cells were cultured in Dulbecco's modified Eagle medium (DMEM) supplemented with 10% fetal calf serum. High efficiency electroporation of cells was done as described²⁰. Reporter assays were carried out using calcium-phosphate transfection of 0.5 □g NF-κB-Luc, 1 ng SV40-Renilla and 2.5 μg PSUPER vectors. Forty-eight hours after transfection cells were stimulated with 200 nM PMA or 20 ng/ml TNF-α and luciferase activity was measured 72 hrs post-transfection. Sodium acetylsalicylic acid (10 mM) or Prostaglandin A1 (8 μM) was added to the cells 48 hrs after transfection, and reporter activity was measured 72 hrs after transfection. In Hela cells NF-κB activity was measured 2 hours after PMA stimulation.

Immunoblotting, Immunoprecipitation and Kinase Assay.

Western blots were performed using whole cell extracts, separated on 8-12% SDS-PAGE gels and transferred to polyvinylidine difuoride membranes (Millipore). Western blots were probed with the indicated antibodies. Transformed human embryonic kidney cells (293 cells) were transfected by calcium-phosphate precipitation with the indicated plasmids, 48 hrs post-transfection cells were lysed in ELB buffer (0.25M NaCL, 0.1% NP-40, 50 mM Hepes pH 7.3) supplemented with “Complete” protease inhibitors (Roche), centrifuged and protein complexes were immunoprecipitated with 2 μg of the indicated antibodies conjugated to protein G sepharose beads. The beads were washed four times with ELB buffer and protein complexes were eluted by boiling in SDS-sample buffer and resolved on 10% SDS-PAGE. Imunoprecipitation/kinase assays were performed essentially as described²¹.

Apoptosis Assays.

Electroporated Hela cells with the indicated plasmids were treated with 200 nM PMA for 2-3 hrs 72 hrs post-transfection followed by 12 hrs incubation in medium containing 10 ng/ml TNF-α and 10 μg/ml cycloheximide. Viable cells were quantified using the trypan-blue exclusion method. Alternatively, apoptotic cells were removed by PBS washing, adherent cells were fixed in 4% paraformaldehyde and stained using 0.1% crystal violet (Sigma) and the optical density at 590 nm was determined as described²². To inhibit NF-κB activity medium was supplemented with 10 mM Sodium acetylsalicylic acid 3.5 hrs before TNF-α addition.

SEQUENCE TABLE
SEQ ID NO:1

GATCCCCGAGCCAGTCGGATGTAGATTTCAAGAGAATCTACATCCGACTG
GCTCTTTTTGGAAA

SEQ ID NO:2

GATCCCCGTAAATTCTGAAGGCGAATTTCAAGAGAATTCGCCTTCAGAAT
TTACTTTTTGGAAA

SEQ ID NO:3

GATCCCCGCCCTCCTAAATCAGGCAATTCAAGAGATTGCCTGATTTAGGA
GGGCTTTTTGGAAA

SEQ ID NO:4

GATCCCCGTTGAGAAATGGAGTGAAGTTCAAGAGACTTCACTCCATTTCT
CAACTTTTTGGAAA

SEQ ID NO:5

GATCCCCCAGTTATATTCTGTGATGTTTCAAGAGAACATCACAGAATATA
ACTGTTTTTGGAAA

SEQ ID NO:6

GATCCCCGAGGTGTTGGGGACAAAGGTTCAAGAGACCTTTGTCCCCAACA
CCTCTTTTTGGAAA

SEQ ID NO:7

GATCCCCGTGGGCTCATTGGCTGAAGTTCAAGAGACTTCAGCCAATGAGC
CCACTTTTTGGAAA

SEQ ID NO:8

GATCCCCGAGCTACTGAGGACAGAAATTCAAGAGATTTCTGTCCTCAGTA
GCTCTTTTTGGAAA

### REFERENCES

1. Wilkinson, K. D. Ubiquitination and deubiquitination: targeting of proteins for degradation by the proteasome. Semin Cell Dev Biol 11, 141-8. (2000).
2. Chung, C. H. & Baek, S. H. Deubiquitinating enzymes: their diversity and emerging roles. Biochem Biophys Res Commun 266, 633-40. (1999).
3. D'Andrea, A. & Pellman, D. Deubiquitinating enzymes: a new class of biological regulators. Crit Rev Biochem Mol Biol 33, 337-52. (1998).
4. Bignell, G. R. et al. Identification of the familial cylindromatosis tumour-suppressor gene. Nat Genet 25, 160-5. (2000).
5. Yin, M. J., Yamamoto, Y. & Gaynor, R. B. The anti-inflammatory agents aspirin and salicylate inhibit the activity of I(kappa)B kinase-beta. Nature 396, 77-80. (1998).
6. Nakamura, T., Hillova, J., Mariage-Samson, R. & Hill, M. Molecular cloning of a novel oncogene generated by DNA recombination during transfection. Oncogene Res 2, 357-70. (1988).
7. Papa, F. R. & Hochstrasser, M. The yeast DOA4 gene encodes a deubiquitinating enzyme related to a product of the human tre-2 oncogene. Nature 366, 313-9. (1993).
8. Li, M. et al. Deubiquitination of p53 by HAUSP is an important pathway for p53 stabilization. Nature 416, 648-53. (2002).
9. Jensen, D. E. et al. BAP1: a novel ubiquitin hydrolase which binds to the BRCA1 RING finger and enhances BRCA1-mediated cell growth suppression. Oncogene 16, 1097-112. (1998).
10. Li, Z. et al. Ubiquitination of a novel deubiquitinating enzyme requires direct binding to von Hippel-Lindau tumor suppressor protein. J Biol Chem 277, 4656-62. (2002).
11. Brummelkamp, T. R., Bernards, R. & Agami, R. A System for Stable Expression of Short Interfering RNAs in Mammalian Cells. Science 296, 550-553. (2002).
12. Karin, M., Cao, Y., Greten, F. R. & Li, Z. W. NF-kappaB in cancer: from innocent bystander to major culprit. Nat Rev Cancer 2, 301-10. (2002).
13. Borodovsky, A. et al. Chemistry-based functional proteomics reveals novel members of the deubiquitinating enzyme family. Chem Biol 9, 1149-59. (2002).
14. Smahi, A. et al. The NF-kappaB signalling pathway in human diseases: from incontinentia pigmenti to ectodermal dysplasias and immune-deficiency syndromes. Hum Mol Genet 11, 2371-5. (2002).
15. Baud, V. & Karin, M. Signal transduction by tumor necrosis factor and its relatives. Trends Cell Biol 11, 372-7. (2001).
16. Rossi, A. et al. Anti-inflammatory cyclopentenone prostaglandins are direct inhibitors of IkappaB kinase. Nature 403, 103-8. (2000).
17. Schmidt-Ullrich, R. et al. NF-kappaB activity in transgenic mice: developmental regulation and tissue specificity. Development 122, 2117-28. (1996).
18. Schmidt-Supprian, M. et al. NEMO/IKK gamma-deficient mice model incontinentia pigmenti. Mol Cell 5, 981-92. (2000);
19. Smahi, A. et al. Genomic rearrangement in NEMO impairs NF-kappaB activation and is a cause of incontinentia pigmenti. The International Incontinentia Pigmenti (IP) Consortium. Nature 405, 466-72. (2000).
20. Agami, R. & Bernards, R. Distinct initiation and maintenance mechanisms cooperate to induce G1 cell cycle arrest in response to DNA damage. Cell 102, 55-66 (2000).
21. Chen, G., Cao, P. & Goeddel, D. V. TNF-induced recruitment and activation of the IKK complex require Cdc37 and Hsp90. Mol Cell 9, 401-10. (2002).
22. Serrano, M., Lin, A. W., McCurrach, M. E., Beach, D. & Lowe, S. W. Oncogenic ras provokes premature cell senescence associated with accumulation of p53 and p161NK4a. Cell 88, 593-602 (1997).
23. Cramer T et al (2003) “Hif-1alpha is essential for myeloid cell-mediated inflammation” Cell Vol. 112(5) pp. 645-657.
24. Lee et al (2002) “vascular growth factors for coronary angiogenesis” J. Interv. Cardiol. Vol 15(6) pp 511-518.
25. Ross J S et al (2001) “Atherosclerosis: a cancer of the blood vessels? ” Am. J. Clin. Pathol. Vol. 116 pp S97-S107.
26. Semenza G (2002) “Signal transduction to hypoxia-inducible factor 1” Biochem. Pharmacol Vol 64(5-6) pp. 993-998.
27. Vagnucci A H et al (2003) “Alzheimer's disease and angiogenesis” Lancet Vol 361(9357) pp 605-608.
28. Witmer et al (2003) “Vascular endothelial growth factors and angiogenesis in eye disease” Prog. Retin. Eye Res. Vol. 22(1) pp 1-29.
29. Dredge et al (2002) “Recent developments in antiangiogenic therapy” Expert Opin. Biol Ther. Vol 2(8) 953-966
30. Healy D L et al (1998) “Angiogenesis: a new theory for endometriosis” Hum Reprod Update Vol 4(5) pp 736-740.

1. An assay method which includes:
bringing into contact a putative modulator and a VDU1 polypeptide;
determining whether the putative modulator binds and/or modulates an activity of VDU1;
determining the effect of the putative modulator on HIF-α stability and/or on the ubiquitination state of HIF-α, in a test system comprising HIF-α and VDU1.

2. An assay method according to claim 1, which includes:
bringing into contact a VDU1 polypeptide with a putative modulator;
determining binding between the VDU1 polypeptide and the putative modulator;
bringing the putative modulator into contact with a test system comprising VDU1 and HIF-α; and
determining the effect of the putative modulator on the stability and/or state of ubiquitination of HIF-α.

3. An assay method according to claim 1 which includes:
bringing into contact a VHL polypeptide, a VDU1 polypeptide and a putative modulator;
determining whether the putative modulator modulates the interaction of the VHL and VDU1 polypeptides;
bringing the putative modulator into contact with a test system comprising VDU1, VHL and HIF-α;
determining the effect of the putative modulator on the stability and/or state of ubiquitination of HIF-α.

4. An assay method according to claim 3, wherein the assay method comprises bringing into contact a VHL polypeptide, a VDU1 polypeptide and a putative modulator compound under conditions where the VHL polypeptide and the VDU1 polypeptide, in the absence of modulator, are capable of forming a complex.

5. An assay method which includes:
bringing a putative modulator into contact with VDU1 and an ubiquitinated VDU1 substrate;
determining the ability of the putative modulator to modulate the stabilisation and/or state of ubiquitination of the substrate by VDU1;
bringing the putative modulator into contact with a test system comprising VDU1 and HIF-α;
determining the effect of the putative modulator on the stability and/or state of ubiquitination of HIF-α.

6. An assay method according to claim 1 in which the test system further comprises VHL.

7. An assay method according to claim 1, wherein the test system is a cell.

8. An assay method according to claim 7, wherein the cell is under hypoxic conditions.

9. An assay method according to claim 7, wherein the cell is under normoxic conditions.

10. An assay method according to claim 7, wherein the effect of the putative modulator on HIF-α stability is determined by the activity of a HIF-responsive reporter gene.

11. An assay method which includes:
bringing into contact a putative modulator with a test system comprising VDU1 and ubiquitinated HIF-α;
determining the ability of the putative modulator to modulate the stabilisation and/or state of ubiquitination of HIF-α by VDU1.

12. An assay method according to claim 11 in which the test system further comprises VHL.

13. An assay method according to claim 1, wherein the putative modulator is brought into contact with the test system under conditions where VDU1 is capable of stabilising HIF-α, in the absence of the modulator.

14. A modulator of VDU1 for use in a method of medical treatment.

15. The modulator according to claim 14 which is an antibody against VDU1.

16. The modulator according to claim 14 which is a nucleic acid comprising a sequence encoding VDU1, such that when the modulator is present in a cell VDU1 expression is enhanced.

17. The modulator according to claim 14 which is an antisense RNA comprising a sequence which hybridises to the VDU1 mRNA, a double stranded VDU1 RNA, or a ribozyme which targets VDU1 RNA, or which is a vector encoding said antisense RNA, double stranded RNA or ribozyme, such that when the modulator is present in a cell VDU1 expression is reduced.

18. The modulator according to claim 14, which is a polypeptide having an amino acid sequence corresponding to a portion of the VHL or VDU1 amino acid sequence, and which binds specifically to VHL or VDU1 to prevent VHL and VDU1 from interacting.

19. Use of a modulator of VDU1 for the manufacture of a medicament for the treatment of a condition in which modulation of HIF is of therapeutic value.

20. (canceled)

21. The use according to claim 19 which comprises use of an inhibitor of VDU1 for the manufacture of a medicament for the treatment of a condition in which inhibition of HIF activity is of therapeutic value.

22. The use according to claim 21, wherein the disease is selected from inflammatory disease, cancer, macular degeneration and diabetic retinopathy, Alzheimer's, atherosclerosis, psoriasis, rheumatoid arthritis and endometriosis.

23. The use according to claim 19, which comprises use of an activator of VDU1 for the manufacture of a medicament for treatment of a condition in which activation of HIF is of therapeutic value.

24. The use according to claim 23, wherein the disease is selected from peripheral and coronary artery disease and myocardial ischaemia.

25. A method of treating a disease in which modulation of HIF is of therapeutic value, the method comprising administering to an individual an effective amount of an agent which modulates the activity of VDU1.

26. A composition comprising a modulator of VDU1 and a pharmaceutically acceptable excipient.

27. A composition comprising a modulator according to claim 15 and a pharmaceutically acceptable excipient.

28. A method of treating an individual with cylindromatosis by administering to the individual an effective amount of an NF-κB inhibitor.

29. The method of claim 28 wherein said inhibitor is aspirin or prostaglandin A1.

30. Use of an NF-κB inhibitor for the manufacture of a medicament for the treatment of cylindromatosis.

31. Use according to claim 30 wherein said inhibitor is aspirin or prostaglandin A1.

32. A method of treating a disease associated with activation of NF-κB which in an individual comprises administering to an individual an effective amount of an agent which increases expression of CYLD.

33. Use of an agent which increases expression of CYLD for the manufacture of a medicament for the treatment of a disease associated with activation of NF-κB.

34. An assay method which includes the steps of:
providing a cell culture in which CYLD activity is suppressed or missing;
bringing the culture into contact with an agent to be assayed; and
determining the effect of the agent on the activity of NF-κB.

35. The method of claim 34 wherein CYLD activity is suppressed using siRNA.

36. The method of claim 34 wherein the effect of the agent is determined using a reporter gene construct.
